(19) United States
(12) Patent Application Publication
(10) Pub. No.: US 2005/0163857 A1
(43) Pub. Date: Jul. 28, 2005

(54) Clarithromycin formulations having improved bioavailability

(76) Inventors: Ashok Rampal, Amritsar, Punjab (IN); Rajeev S Raghuvanshi, Delhi (IN); Manoj Kumar Paruthi, Haryana India (IN)

Correspondence Address: Jayadeep R Deshmukh;Ranbaxy Pharmaceutical Inc, 600 College Road East, Suite 2100, Princeton, NJ, 08540, US

(21) Appl. No.: 10/509,704

(22) PCT Filed: Apr. 3, 2003
(86) PCT No.: PCT/IB03/01223

(30) Foreign Application Priority Data
Apr. 3, 2002 (IN) 425/DEL/2002

(51) Int. Cl. A61K 9/14; A61K 31/7052

(52) U.S. Cl. 424/489; 514/28

(57) ABSTRACT

A pharmaceutical composition includes micronized clarithromycin and exhibits improved dissolution characteristics relative to a pharmaceutical composition that includes unmicronized clarithromycin. The clarithromycin may have a particle size less than approximately 35 microns. One process for preparing an extended release tablet of the clarithromycin includes micronizing the clarithromycin; blending the micronized clarithromycin with one or more rate controlling polymers and pharmaceutically acceptable excipients; granulating the blend; and compressing to form a tablet. To treat a bacterial infection in a mammal in need of treatment, a patient may be administered a pharmaceutical composition that includes micronized clarithromycin.

### TECHNICAL FIELD OF THE INVENTION

The technical field of the invention relates to solid pharmaceutical compositions of clarithromycin with enhanced absorption and dissolution characteristics provided by micronizing the clarithromycin.

### BACKGROUND OF THE INVENTION

There is an ever-present need in the pharmaceutical industry for improved pharmaceutical formulations that enhance the efficacy of poorly soluble therapeutic agents. There is especially a need for formulations that (1) enhance the absorption of poorly soluble therapeutic agents, and (2) extend the period of duration of effect of the therapeutic agents.

The aqueous solubility of drug substances plays an important role in the formulation of dosage forms. For the oral route of administration it is well experienced that, unless the substance has an aqueous solubility above 10 mg/ml over the pH range 1-7, potential absorption problems may occur. Numerous active ingredients suffer from the disadvantage of being poorly soluble in an aqueous medium, thus having an insufficient dissolution profile and consequently, poor bioavailability following oral administration. The therapeutic dose required to be administered must be increased in order to obviate this disadvantage. This necessitates the administration of active ingredients three or four times a day in order to achieve the desired effect.

For a drug that is administered in multiple doses, it is reported that the patient compliance is as high as 87% when administered once a day as when compared to 39% for a q.i.d. dosage regimen. An extended-release dosage form may improve the quality of therapy and the safety profile relative to a conventional dosage form. However, in order to be effective, these extended release formulations should completely release the drug within a predetermined period.

Erythromycin and its derivatives are useful in treating bacterial infections and are known as anti-bacterial agents useful against a number of organisms and are typically administered two to three times a day as immediate release compositions. In particular, 6-0-methoxyerythromycin A (clarithromycin), which has been disclosed in U.S. Pat. No. 4,331,803, has to be administered at least twice daily for optimal effect.

### SUMMARY OF THE INVENTION

In one general aspect, there is provided a pharmaceutical composition which includes micronized clarithromycin and exhibits improved dissolution characteristics relative to a pharmaceutical composition that includes unmicronized clarithromycin.

Embodiments of the pharmaceutical composition may include one or more of the following features. For example, the clarithromycin may have a particle size less than approximately 50 microns, and more particularly, less than 35 microns. The clarithromycin in the pharmaceutical composition may make up between approximately 100 mg and approximately 1000 mg of the pharmaceutical composition. The pharmaceutical formulation may be an extended release formulation.

The pharmaceutical composition may further include one or more rate controlling polymers. The rate controlling polymers may be one or more of carbohydrate gums, polyuronic acid salts, cellulose ethers, and acrylic acid polymers. The carbohydrate gums may be one or more of xanthan gum, tragacanth gum, gum karaya, guar gum, acacia, gellan, and locust bean gum. The polyuronic acid salts may be one or more of alkali metal salts of alginic acid and pectic acid. The cellulose ethers may be one or more of hydroxypropyl methylcellulose, hydroxypropyl cellulose, and carboxymethyl cellulose. The acrylic polymers may be the acrylic polymer available under the brand name carbopol.

The pharmaceutical composition may further include one or more pharmaceutically acceptable excipients. The pharmaceutically acceptable excipients may be one or more of gas generating components, swelling agents, lubricants, and fillers.

The pharmaceutical composition may be a once a day formulation. The dosage form may be a tablet or a capsule.

The clarithromycin may be micronized in air jet mill, and may be co-micronized with one or more pharmaceutical inert carriers. The pharmaceutically inert carrier may be one or more cellulose derivatives, silicate derivatives, and clays. The cellulose derivative may be one or more of microcrystalline cellulose and carboxymethyl cellulose. The silicate derivative may be one or more of magnesium silicate, colloidal silicon dioxide, magnesium trisilicate, and magnesium aluminicum silicate. The clay may be one or more of veegum and bentonite. The amount of pharmaceutically inert carrier may be between approximately 2% and approximately 25% by weight relative to the total weight of the pharmaceutical composition.

The pharmaceutical composition may exhibit improved absorption characteristics relative to a pharmaceutical composition that includes unmicronized clarithromycin. The pharmaceutical composition has an area-under-the-curve (AUC) comparable to the area-under-the-curve (AUC) of a twice-daily immediate release dosage form.

The pharmaceutical composition may further include one or more of omeprazole, metronidazole, amoxicillin, rifampicin, lansoprazole, ciprofloxacin, ethambutol, and ritonavir. The clarithromycin and the one or more active ingredients may be combined in a single pharmaceutical composition.

The pharmaceutical composition may further include unmicronized clarithromycin, thereby forming a mixture of unmicronized and micronized clarithromycin.

In another general aspect, there is provided a process for preparing an extended release tablet of clarithromycin which includes micronizing clarithromycin; blending the micronized clarithromycin with one or more rate controlling polymers and pharmaceutically acceptable excipients; granulating the blend; and compressing to form a tablet.

Embodiments of the process may include one or more of the following features. For example, the clarithromycin may be micronized to have a particle size less than approximately 50 microns. More particularly, the clarithromycin may be micronized to have a particle size less than 35 microns. The clarithromycin may make up between approximately 100 mg and approximately 1000 mg of the tablet.

In the process, the clarithromycin may be micronized in an air jet mill and micronizing may include co-micronizing the clarithromycin with one or more pharmaceutical inert carriers. The pharmaceutically inert carrier may be one or more cellulose derivatives, silicate derivatives, and clays.

In another general aspect, there is provided a method of treating a bacterial infection in a mammal in need of treatment which includes administering a pharmaceutical composition comprising micronized clarithromycin and one or more pharmaceutically acceptable excipients.

The clarithromycin in the pharmaceutical composition taken to provide antibacterial activity may include at least some clarithromycin that has been micronized to have a particle size less than 50 microns, and more particularly, less than 35 microns, and the clarithromycin may make up between approximately 100 mg and approximately 1000 mg of the pharmaceutical composition. The method of treating may further include administering one or more of omeprazole, metronidazole, amoxicillin, rifampicin, lansoprazole, ciprofloxacin, ethambutol, and ritonavir with the micronized clarithromycin.

The details of one or more embodiments of the invention are set forth in the description below. Other features, objects, and advantages of the invention will be apparent from the description and claims.

### DETAILED DESCRIPTION OF THE INVENTION

The process of developing pharmaceutical compositions of clarithromycin is challenging for the pharmaceutical formulator because of opposing solubility and stability constraints. In particular, clarithromycin has increased solubility but reduced stability at the acidic pH conditions in the stomach, and increased stability but reduced solubility at the alkaline pH of the lower portion of the intestine (pH 6.0 to 8.0). These constraints result in poor bioavailability of clarithromycin. In spite of these competing constraints, the inventors nonetheless realized the desirability of dosage forms of clarithromycin having improved dissolution and absorption characteristics that can be administered once per day, and conducted research and development activities for developing such a clarithromycin formulation. As a result of these efforts, the inventors have surprisingly found that the dissolution and absorption characteristics of clarithromycin, as well as its bioavailability, can be increased by micronizing the clarithromycin.

The term “micronization” used herein means any process or methods by which the size of the particles is reduced. As also used herein, clarithromycin particles with reduced size are referred to as “micronized particles of clarithromycin” or “micronized clarithromycin”.

The clarithromycin used in the pharmaceutical compositions described herein can be prepared by any known method, such as, for example, using either of the procedures disclosed in U.S. Pat. No. 4,331,803 or U.S. Pat. No. 4,672,109. Both of these patents are incorporated herein in their entirety by reference.

The process of the invention for preparing a solid formulation of clarithromycin with improved dissolution and absorption characteristics includes the micronization of clarithromycin. Size reduction, or micronization, may be carried out using any of the conventionally known mills, such as a ball mill, colloid mill, grinding mill, air jet mill, roller mill, impact mill, etc. Air jet milling is particularly well suited for this application as it is a well proven technique that consistently produces particles of a size less than 35 microns. Primary advantages of air jet milling are that the predominant particle size reduction occurs through particle to particle collisions, there is limited particle size reduction that results from metal to product contact, and there is no generation of heat that can adversely affect the particles being micronized.

The process of air jet milling involves exposing the material to be micronized to streams of compressed air or gas. Particles in the fluidized bed created by the gas streams are accelerated towards the center of the mill and collide with the slower moving particles. These collisions break the particles into smaller particles, thereby micronizing the particles. The air jet mills operate by applying opposing air flows and centrifugal forces. By balancing the two forces, desired particle size and fines can be separated.

The reduction of the particle size of clarithromycin to a D₉₀ particle size of less than 50 microns, and more particularly less than 35 microns, results in improved bioavailability of clarithromycin pharmaceutical compositions as compared to clarithromycin pharmaceutical compositions that contain larger sized clarithromycin particles. Clarithromycin particles having a D₉₀ particle size of less than about 50 microns, and more particularly less than about 35 microns, are referred to herein as “micronized clarithromycin particles.” As used herein, “D₉₀ particle size” is the particle size of at least 90% of the particles of clarithromycin used in the composition.

When clarithromycin is micronized, the resulting particles can be difficult to process because highly micronized particles may possess poor flow properties and have a tendency to agglomerate during processing. To overcome these potential and actual difficulties, the clarithromycin may be micronized in the presence of one or more pharmaceutically inert carrier(s) or mixed with inert carriers after micronization to neutralize the static charge.

As used herein, the term “pharmaceutically inert carrier” refers to a substance that is physiologically acceptable, compatible with the drug and other excipients in the formulation, and has a capacity to adsorb the drug on its surface. Carriers prevent reagglomeration of drug particles and also help in wetting of the drug by uptake of water by capillary action and thereby enhancing drug dissolution further.

The pharmaceutically inert carrier may be selected from cellulose derivatives such as microcrystalline cellulose and carboxymethyl cellulose; silicate derivatives such as magnesium silicate, colloidal silicon dioxide, magnesium trisilicate, and magnesium aluminum silicate; and clays such as veegum, bentonite, etc.

The micronized clarithromycin, micronized either with or without an inert carrier, then is processed to form a solid formulation and finished dosage form (e.g., tablet or capsule) that includes a rate controlling polymer and one or more pharmaceutically acceptable excipients. The rate controlling polymer provides sustained or extended release characteristics to the finished dosage form such that a patient can reduce the number of times per day that they must take clarithromycin to once or twice per day. For example, the amount of micronized clarithromycin in the finished dosage form can be present at between approximately 100 mg and 1000 mg and the finished dosage form taken only once per day. When the clarithromycin is micronized, it exhibits improved dissolution and absorption characteristics relative to marketed clarithromycin formulations.

The rate-controlling polymers of the solid formulation and finished dosage form may be selected from the group that includes carbohydrate gum, polyuronic acid salts, cellulose ethers, acrylic acid polymers and mixtures, thereof. Carbohydrate gums may be selected from the group that includes xanthan gum, tragacanth gum, gum karaya, guar gum, acacia, gellan, locust bean gum and other carbohydrate gums having similar properties. Polyuronic acid salts include alkali metal salts of alginic acid or pectic acid and mixtures thereof. Examples of alkali metal salts of alginic acid that may be used include sodium alginate, potassium alginate, ammonium alginate and other suitable alkali metal salts of alginic acid. Cellulose ethers include hydroxypropyl methyl cellulose, hydroxypropyl cellulose and other suitable cellulose ethers. Any suitable polyacrylic acid polymer, such as is available under the brand name carbopol, may be used.

The other pharmaceutically acceptable excipients include gas generating components, swelling agents, lubricants, binders, and fillers and diluents. Gas generating components include carbonates, such as calcium carbonate; bicarbonates such as sodium bicarbonate; sulfites such as sodium sulfite; and other suitable known gas generating components. Swelling agents include cross-linked polyvinylpyrrolidone, cross-linked carboxymethyl cellulose sodium, sodium starch glycolate and other suitable, known swelling agents. Lubricants include talc, calcium stearate, magnesium stearate, polyethylene glycols, silicon dioxide, sodium lauryl sulphate, sodium stearyl fumarate, other suitable, known lubricants, and mixtures thereof. Binders include polyvinyl pyrrolidone (PVP) and other suitable, known binders. Fillers and diluents include lactose and other suitable, known fillers and diluents.

The following examples are provided to illustrate various implementations of the invention without being limiting.

### EXAMPLE 1

PREPRATION OF EXTENDED RELEASE TABLET
FORMULATION OF CLARITHROMYCIN

| Ingredients | Mg/tablet |
|---|---|
| Clarithromycin micronized (Particle size: | 1000.0 |
| D₉₀ equivalent to 31.93 microns) | |
| Hydroxypropyl methylcellulose K15M | 10.0 |
| Hydroxypropyl methylcellulose K4M | 17.5 |
| Polyvinyl pyrrolidone K-30 | 25.0 |
| Lactose | 50.0 |
| Magnesium stearate | 12.5 |
| Talc | 10.0 |
| Sodium stearyl fumarate | 20.0 |
| Colloidal silicon dioxide | 5.0 |
| Total weight | 1150.0 |

Micronized clarithromycin, hydroxypropyl methylcellulose K15M, hydroxypropyl methylcellulose K4M, polyvinyl pyrrolidone K30 and lactose were sieved through a British Standard Sieve (BSS) 44 mesh sieve, blended together, and granulated with water. The resulting granulate was dried in a fluid bed drier at 60° C. for 20 minutes. The dried granules were sifted through a BSS 16 mesh sieve. The granules obtained were lubricated with the remaining ingredients and compressed to tablets.

The clarithromycin of the tablet of Example 1 was not micronized with an inert carrier. Nonetheless, a portion of the colloidal silicon dioxide, which is described herein as being a suitable inert carrier for co-micronization, of Example 1 could have been provided for co-micronizing with clarithromycin and the remainder later added for lubrication.

### EXAMPLE 2

Table 1 illustrates the effect of particle size on the in-vitro drug release profile of an extended release clarithromycin tablet. The extended release tablets were prepared according to the composition of Example 1 using two different particle sizes, one micronized (D₉₀=29.73 microns) and another unmicronized (D₉₀=246.39 microns). As illustrated in Table 1, the micronized clarithromycin formulation provided a significantly improved dissolution profile relative to an unmicronized clarithromycin formulation. The dissolution was carried out in 1000 ml mixed phosphate buffer of pH 4.0, at 80 rpm using USP Apparatus II with 10 mesh sinker basket and the paddle height was adjusted to 4.5 cm from the bottom of the basket.

TABLE 1
Dissolution profile of clarithromycin extended release pharmaceutical
compositions prepared with clarithromycin particles of different sizes
carried out in USP apparatus II/1000 ml/pH 4.0, mixed phosphate
buffer/80 rpm.

| | |
|---|---|
| | Percent (%) drug released |
| Time | Particle size |

| (hr) | D₉₀ = 29.73 microns | D₉₀ = 246.39 microns |
|---|---|---|
| 1 | 14 | 12 |
| 2 | 30 | 20 |
| 4 | 59 | 33 |
| 8 | 99 | 59 |
| 10 | 103 | 70 |

### EXAMPLE 3

Bioavailability study: The extended release clarithromycin solid formulation of Example 1 having clarithromycin with a mean particle diameter of 31.93 microns was compared to commercially available tablets (Abbott Laboratories Biaxin film tab 500 mg b.i.d.) in a bioavailability study. The bioavailability study was performed on six healthy subjects. It was conducted as a single dose, open, randomized, balanced, crossover study, under fed conditions. Blood samples were drawn at selected times following each treatment. Blood levels of the drug for both the test and the reference drugs were determined and compared for the two critical parameters: Area Under the plasma concentration—time Curve (AUC) and Maximum plasma concentration (Cmax). The results, shown in Table 2, illustrate the substantially similar bioavailability of an extended release clarithromycin formulation compared to a conventional, twice daily formulation.

Test Drug: Extended release clarithromycin formulation made according to Example 1 and comprising clarithromycin with a particle size of D₉₀ equivalent to 31.93 microns.

Reference Drug: Commercially available clarithromycin formulations (Abbott Laboratories, Biaxin Filmtab 500 mg) administered twice daily.

TABLE 2
Comparison of bioavailabiity of extended release micronized
clarithromycin formulation and conventional twice daily
clarithromycin formulation.

| Formulation | | AUC 0-24 h (μg · hr/ml) | T/R |
|---|---|---|---|
| Biaxin Filmtab | b.i.d | 37.95 | — |
| 500 mg (R) | First dose | 16.9 | — |
| Clarithromycin | 1000 mg | 36.6 | 96.4% |
| XL (T) | Interpolated value | 18.3 | 108.3% |
| | for 500 mg | | |

### EXAMPLE 4

Example 4 involves preparing an extended release pharmaceutical formulation of clarithromycin that has a mean particle size of D₉₀ equivalent to 275.58 microns.

| Ingredients | Mg/tablet |
|---|---|
| Clarithromycin | 500 |
| Lactose | 117 |
| Hydroxypropyl cellulose-L | 105 |
| Hydroxypropyl cellulose-M | 125 |
| Polyvinyl pyrrolidone K 30 | 10 |
| Talc | 18 |
| Sodium stearyl fumarate | 18 |
| Colloidal silicon dioxide | 2 |
| Magnesium stearate | 5 |

Unmicronized clarithromycin, hydroxypropyl cellulose M, hydroxypropyl cellulose L, polyvinyl pyrrolidone K30 and lactose were sieved through a British Standard Sieve (BSS) 44 mesh sieve, blended together, and granulated with water. The resulting granulate was dried in a fluid bed drier at 60° C. for 20 minutes. The dried granules were sifted through a BSS 16 mesh sieve. The granules obtained were lubricated with the remaining ingredients and compressed to form tablets.

The extended release clarithromycin formulation of Example 4 having clarithromycin with a mean particle size of D₉₀ equivalent to 275.58 microns was compared to commercially available tablets (Abbott Laboratories Klaricid XL tablets, 500 mg) in a bioavailability study. The study was conducted on twelve healthy subjects as single dose, open, randomized, balanced crossover study. The results of the study are presented in Table 3. These results indicate the reduced bioavailability of unmicronized clarithromycin tablets relative to commercially available clarithromycin extended release tablets. The results further support the discovery that micronizing clarithromycin provides improved bioavailability and that to provide the bioavailability of conventional clarithromycin formulations the clarithromycin should be micronized.

Test Drug: Extended release clarithromycin formulation (500 mg) made according to Example 4 and comprising clarithromycin with a particle size of D₉₀ equivalent to 275.58 microns.

Reference Drug: Commercially available clarithromycin extended release tablets (Klaricid XL 500 mg)

TABLE 3

AUC 0-24 h (μg · hr/ml)

| Test | Reference | T/R |
|---|---|---|
| 13.0 | 17.72 | 73.36% |

While several particular forms of the invention have been illustrated and described, it will be apparent that various modifications and combinations of the invention detailed in the text can be made without departing from the spirit and scope of the invention. For example, the clarithromycin used in the pharmaceutical compositions does not necessarily need to include only micronized clarithromycin but instead can be made up of a mixture of micronized and unmicronized clarithromycin, e.g., a first batch of clarithromycin is micronized and then mixed with a second batch of clarithromycin which has not been micronized. Moreover, the micronized clarithromycin may be administered with (e.g., as a single pharmaceutical combination composition, simultaneously, or within a short time) other drugs and drug products to treat conditions that may be related to or that occur concurrently with a condition that involves the treatment of a bacterial infection using clarithromycin. Such drugs that may be co-administered with the micronized clarithromycin generally include one or more of omeprazole, metronidazole, amoxicillin, rifampicin, lansoprazole, ciprofloxacin, ethambutol, and ritonavir. For example, the combinations may include a single pharmaceutical composition or joint administration of: (1) omeprazole, metronidazole, and clarithromycin; (2) omeprazole, amoxicillin, and clarithromycin; (3) rifampicin and clarithromycin; (4) lansoprazole and clarithromycin; (5) ciprofloxacin and clarithromycin; (6) lansoprazole, amoxicillin, and clarithromycin; and (7) ethambutol, ritonavir, and clarithromycin.

Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. A pharmaceutical composition comprising micronized clarithromycin, wherein the pharmaceutical composition exhibits improved dissolution characteristics relative to a pharmaceutical composition that includes unmicronized clarithromycin.

2. The pharmaceutical composition of claim 1, wherein the clarithromycin has a particle size less than 50 microns.

3. The pharmaceutical composition of claim 1, wherein the clarithromycin has a particle size less than 35 microns.

4. The pharmaceutical composition of claim 1, wherein the clarithromycin comprises between approximately 100 mg and approximately 1000 mg.

5. The pharmaceutical composition of claim 1, wherein the pharmaceutical formulation comprises an extended release formulation.

6. The pharmaceutical composition of claim 1, further comprising one or more rate controlling polymers.

7. The pharmaceutical composition of claim 6, wherein the rate controlling polymers comprises one or more of carbohydrate gums, polyuronic acid salts, cellulose ethers, and acrylic acid polymers.

8. The pharmaceutical composition of claim 7, wherein the carbohydrate gums comprise one or more of xanthan gum, tragacanth gum, gum karaya, guar gum, acacia, gellan, and locust bean gum.

9. The pharmaceutical composition of claim 7, wherein the polyuronic acid salts comprise one or more of alkali metal salts of alginic acid and pectic acid.

10. The pharmaceutical composition of claim 7, wherein the cellulose ethers comprise one or more of hydroxypropyl methylcellulose, hydroxypropyl cellulose, and carboxymethyl cellulose.

11. The pharmaceutical composition of claim 7, wherein the acrylic polymers comprise the acrylic polymer available under the brand name carbopol.

12. The pharmaceutical composition of claim 1, further comprising one or more pharmaceutically acceptable excipients.

13. The pharmaceutical composition of claim 12, wherein the pharmaceutically acceptable excipients comprise one or more of gas generating components, swelling agents, lubricants, and fillers.

14. The pharmaceutical composition of claim 1, wherein the pharmaceutical composition comprises a once a day formulation.

15. The pharmaceutical composition of claim 1, wherein the dosage form comprises a tablet or a capsule.

16. The pharmaceutical composition of claim 1, wherein the clarithromycin is micronized in air jet mill.

17. The pharmaceutical composition of claim 1, wherein the clarithromycin is co-micronized with one or more pharmaceutical inert carriers.

18. The pharmaceutical composition of claim 17, wherein the pharmaceutically inert carrier comprises one or more cellulose derivatives, silicate derivatives, and clays.

19. The pharmaceutical composition of claim 18, wherein the cellulose derivative comprises one or more of microcrystalline cellulose and carboxymethyl cellulose.

20. The pharmaceutical composition of claim 18, wherein the silicate derivative comprises one or more of magnesium silicate, colloidal silicon dioxide, magnesium trisilicate, and magnesium aluminicum silicate.

21. The pharmaceutical composition of claim 18 wherein clay comprises one or more of veegum and bentonite.

22. The pharmaceutical composition of claim 17, wherein the amount of pharmaceutically inert carrier comprises between approximately 2% and approximately 25% by weight relative to the total weight of the pharmaceutical composition.

23. The pharmaceutical composition of claim 1, wherein the pharmaceutical composition exhibits improved absorption characteristics relative to a pharmaceutical composition that includes unmicronized clarithromycin.

24. The pharmaceutical composition of claim 1, wherein the pharmaceutical composition comprises an area-under-the-curve (AUC) comparable to the area-under-the-curve (AUC) of a twice-daily immediate release dosage form.

25. The pharmaceutical composition of claim 1, further comprising one or more of active ingredients, wherein the active ingredients comprise one or more of omeprazole, metronidazole, amoxicillin, rifampicin, lansoprazole, ciprofloxacin, ethambutol, and ritonavir.

26. The pharmaceutical composition of claim 25, wherein the clarithromycin and the one or more active ingredients are combined in a single pharmaceutical composition.

27. The pharmaceutical composition of claim 1, wherein the pharmaceutical composition further comprises unmicronized clarithromycin.

28. A process for preparing an extended release tablet of clarithromycin, the process comprising:
micronizing clarithromycin;
blending the micronized clarithromycin with one or more rate controlling polymers and pharmaceutically acceptable excipients;
granulating the blend; and
compressing to form a tablet.

29. The process of claim 28, wherein the clarithromycin is micronized to have a particle size less than 50 microns.

30. The process of claim 28, wherein the clarithromycin is micronized to have a particle size less than 35 microns.

31. The process of claim 28, wherein the clarithromycin comprises between approximately 100 mg and approximately 1000 mg of the tablet.

32. The process of claim 28, wherein micronizing comprises micronizing the clarithromycin in an air jet mill.

33. The process of claim 28, wherein micronizing comprises co-micronizing the clarithromycin with one or more pharmaceutical inert carriers.

34. The process of claim 33, wherein the pharmaceutically inert carrier comprises one or more cellulose derivatives, silicate derivatives, and clays.

35. A method of treating a bacterial infection in a mammal in need of treatment, the method comprising administering a pharmaceutical composition comprising micronized clarithromycin and one or more pharmaceutically acceptable excipients.

36. The method of claim 35, wherein the clarithromycin comprises at least some clarithromycin that has been micronized to have a particle size less than 50 microns.

37. The method of claim 35, wherein the clarithromycin comprises at least some clarithromycin that has been micronized to have a particle size less than 35 microns.

38. The method of claim 35, wherein the clarithromycin comprises between approximately 100 mg and approximately 1000 mg of the pharmaceutical composition.

39. The method of claim 35, further comprising administering one or more of omeprazole, metronidazole, amoxicillin, rifampicin, lansoprazole, ciprofloxacin, ethambutol, and ritonavir with the micronized clarithromycin.
